(19) United States
(12) Patent Application Publication
(10) Pub. No.: US 2007/0031872 A1
(43) Pub. Date: Feb. 8, 2007

(54) A141S and G399S mutation in the Omi/HtrA2 protein in Parkinson's disease

(76) Inventors: Rejko Krueger, Tuebingen (DE); Karsten Strauss, Tuebingen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/493,369

(22) Filed: Jul. 25, 2006

Related U.S. Application Data
(63) Continuation of application No. PCT/EP05/00503, filed on Jan. 20, 2005.

(30) Foreign Application Priority Data
Jan. 27, 2004 (DE) 10 2004 004 924.6

(51) Int. Cl. C12Q 1/68; A01K 67/00; C07H 21/04; C12P 19/34; C07K 14/705

(52) U.S. Cl. 435/6; 435/91.2; 800/9; 435/325; 530/350; 536/23.5

(57) ABSTRACT

The present invention relates to a method for diagnosing Parkinson's disease in a human being; nucleic acid molecules used in this method; a nucleic acid molecule which encodes a human Omi/HtrA2 protein which has a genetic modification at amino acid position 141 and/or 399 compared with the wild type, and for corresponding segments thereof; a host, preferably a transgenic non-human mammal, into which such a nucleic acid molecule has been introduced; a (poly)peptide which is encoded by such a nucleic acid molecule; a method for finding substances which bind to Omi/HtrA2 protein which is genetically modified compared with the wild type; a substance found with the aid of this method, and a preferably pharmaceutical composition which comprises such a substance. This invention additionally relates to a kit which comprises at least one of the aforementioned nucleic acid molecules.

### CROSS-REFERENCES TO RELATED APPLICATIONS

This application is a continuation of copending International Patent Application PCT/EP2005/000503 filed on Jan. 20, 2005 and designating the United States, which was not published under PCT Article 21(2) in English, and claims priority of German Patent Application DE 10 2004 004 924.6, filed on Jan. 27, 2004, which is incorporated herein by reference.

### BACKGROUND OF THE INVENTION

1. Field of the Invention

The present invention relates to a method for diagnosing Parkinson's disease in a human being; nucleic acid molecules used in this method; a nucleic acid molecule which encodes a human Omi/HtrA2 protein which has a genetic modification at amino acid position 141 and/or 399 compared with the wild type, and for corresponding segments thereof; a host, preferably a transgenic non-human mammal, into which such a nucleic acid molecule has been introduced; a (poly)peptide which is encoded by such a nucleic acid molecule; a method for finding substances which bind to Omi/HtrA2 protein which is genetically modified compared with the wild type; a substance found with the aid of this method, and a preferably pharmaceutical composition which comprises such a substance. This invention additionally relates to a kit which comprises at least one of the aforementioned nucleic acid molecules.

2. Related Prior Art

Methods of the aforementioned type are generally known in the prior art.

Parkinson's disease is the second commonest neurodegenerative disorder in humans and the second commonest neurological disorder of people of advanced age. 4% of those over eighty are affected, and in Germany alone there are about 250 000 Parkinson's patients.

The cardinal symptoms of Parkinson's disease are tremor, rigidity and akinesia, i.e. trembling which is most pronounced at rest, slowing and paucity of movement, difficulties in initiating movements, shuffling gait with stooping, dulled postural reflexes with tendency to falls, mask-like face, soft, monotonous and stammering speech. About 50% of the patients develop dementia.

Parkinson's disease is moreover associated with a degeneration of the dopamine-producing cells in the substantia nitra pars compacta, presumably via an apoptotic cell death, leading to a functional dysbalance of downstream nuclei. Since dopamine inhibits the activity of nerve cells in various areas of the brain, the loss of dopamine leads to hyperstimulation of these regions of the brain.

The etiology of Parkinson's disease is substantially unknown. Environmental factors such as, for example, pesticides, are suggested to be factors influencing the onset of this disease.

On the other hand, genetic factors play a significant part in the development of Parkinson's disease. These ideas are based on studies on twins (cf. in this connection Piccini et al. (1999), The role of inheritance in sporadic Parkinson's disease: evidence from a longitudinal study of dopaminergic function in twins. Ann. Neurol., 45, 577-582), the identification of large families affected by Parkinson's disease (cf. in this connection for example Nicholl et al., (2002), Two large British kindreds with familial Parkinson's disease: a clinico-pathological and genetic study. Brain, 125, 44-57), and the elevated risk of developing Parkinson's disease in relatives of so-called index patients (cf. in this connection Elbaz et al. (1999), Familial aggregation of Parkinson's disease: a population-based case-control study in Europe. EUROPARKINSON Study Group. Neurology, 52, 1876-1882).

It has to date been possible to identify ten gene loci connected with Parkinson's disease. A review of this is to be found in Krüger et al. (2002), Parkinson's disease: one biochemical pathway to fit all genes?, TRENDS in Molecular Medecine, Vol. 8 No. 5, 236-240. It was further possible to identify four genes, α-Synuclein, Synphilin-1, Parkin and UCH-L1, which are present in mutated form in Parkinson's disease with autosomal dominant or autosomal recessive inheritance. All the gene products of these genes are members of the proteasome protein degeneration degradation pathway. Whereas mutations in the α-synuclein, synphilin-1 and in the UCH-L1 genes are extremely rare, mutations in the Parkin gene are present in almost one half of the cases of autosomal recessive forms of the so-called early onset Parkinson's disease (AR-EOPD); cf. in this connection Lucking et al. (2000), Association between early-onset Parkinson's disease and mutations in the parkin gene. French Parkinson's Disease Genetics Study Group. N. Engl. J. Med., 342, 1560-1567.

It has recently been possible to assign two gene loci which correlate with AR-EOPD, PARK6 and PARK7, to the human chromosome 1p35-36. Whereas it has not yet been possible to identify the underlying gene defect of PARK6, Bonifati et al. were able to identify a homozygous L166P substitution and a deletion in two unrelated families in the DJ-1 gene as a cause of PARK7-associated Parkinson's disease; cf. Bonifati et al. (2003), Mutations in the DJ-1 gene associated with autosomal recessive early-onset parkinsonism. Science 299, 256-259.

In addition, the as yet unpublished German patent application with the file number 103 48 904.5 describes a further new mutation, associated with AR-EOPD in the DJ-1 gene, by which there is an exchange of a glutamic acid molecule for an aspartic acid molecule at amino acid position 64 in the DJ-1 protein.

Diagnosis of the development of Parkinson's disease or AR-EOPD has to date been made essentially by means of the observed cardinal symptoms, which is associated with corresponding uncertainties and risks of misdiagnoses. Such a diagnosis can be confirmed only post mortem by neuropathological detection of the characteristic so-called Lewy bodies, which are abnormal protein deposits in the brain. A predisposition to a corresponding development can therefore at present be diagnosed only with a very large uncertainty factor or not at all. For this reason, the basis for an appropriately targeted preventive treatment of the people affected, both medicinally and, where appropriate, psychotherapeutically, is lacking in the art.

### SUMMARY OF THE INVENTION

It is thus an object of the present invention to provide a method with which the aforementioned prior art disadvantages are avoided. It is intended in particular to provide such a method by means of which a molecular-biological differential diagnosis of the development of or a predisposition to Parkinson's disease is possible for the people affected, which method can be incorporated for example into additional detection of genetic modifications which have already been described in the art as correlated with Parkinson's disease.

This object is achieved by the provision of a method for diagnosing Parkinson's disease in a human organism, which comprises the following steps: (a) provision of a biological sample of the organism; (b) analysis of the biological sample for the presence of a nucleic acid molecule and/or of a (poly)peptide, and (c) correlation of a positive finding with the development of Parkinson's disease and/or with a predisposition to the development of Parkinson's disease, where in step (b) the biological sample is analyzed for the presence of a nucleic acid molecule which encodes an Omi/HtrA2 protein which is genetically modified compared with the wild type, or for segments thereof. The biological sample is preferably analyzed for the presence of a nucleic acid molecule which encodes a human Omi/HtrA2 protein which has a genetic modification at amino acid position 141 and/or 399 compared with the wild type, and for corresponding segments thereof.

The object underlying the invention is completely achieved thereby.

The inventors have surprisingly been able to show for the first time that a genetic modification in the Omi/HtrA2 protein, preferably one at amino acid position 141 or 399, correlates with Parkinson's disease. This realization is all the more surprising since the Omi/HtrA2 gene or the protein encoded thereby has not previously been thought to be connected with Parkinson's disease. The Omi/HtrA2 protein is a serine protease which was described for the first time in 2000; cf. Faccio et al. (2000), Characterization of a novel human serine protease that has extensive homology to bacterial heat shock endoprotease HtrA and is regulated by kidney ischemia, The Journal of Biological Chemistry, Vol. 275 No. 4, pages 2581-2588; Grey et al. (2000), Characterization of human HtrA2, a novel serine protease involved in the mammalian cellular stress response, European Journal of biochemistry Vol. 267, pages 5699-5710. The sequence of the human Omi/HtrA2 gene can be found in the NCBI database: NM_{—}013247.3 or NM_{—}145074.1 (in each case mRNA); AC005041.2 (genomic sequence, BAC); AF141305.1 or AF020760.2 (each cDNA and translation product). All before identified data base entries are incorporated herein by reference. The sequences registered under AF141305.1 are depicted in the enclosed sequence listing under SEQ ID No. 22 (cDNA) and SEQ ID No. 23 (translation product or protein, respectively).

The Omi/HtrA2 protein is a member of the HtrA protease chaperone family and is localized in the intermembrane space of the mitochondria. Omi/HtrA2 is thought to be associated with stress-induced cell death (apoptosis), it having been possible to show that the serine protease activity of the Omi/HtrA2 protein is responsible for mediating caspase independent cell death; cf. Cilenti et al. (2003), Characterization of a novel and specific inhibitor for the pro-apoptotic protease Omi/HtrA2, Journal of Biological Chemistry, Vol. 278, pages 11489-11494. In addition, the Omi-HtrA2 protein is closely connected, via the interaction with the ubiquitin E3 ligase XIAP, with the ubiquitin-mediated protein degradation pathway; cf., for example, Suzuki et al. (2001), A serine protease, HtrA2, is released from the mitochondria and interacts with XIAP, inducing cell death, Molecular Cell, Vol. 8, pages 613-621.

Jones et al. (2003), Loss of Omi mitochondrial protease activity causes the neuromuscular disorder of mnd2 mutant mice, Nature, Vol. 425, pages 721-727, describe the first mutation found in the Omi-HtrA2 gene, through which a serine molecule is exchanged for a cysteine molecule at amino acid position 276 in the protein. The mutation described therein was identified in a mouse mutant which has been known since 1990 and is called mnd2 and which shows neuromuscular impairments and mitochondrial defects. The authors thereof were able to show, in experiments on embryonic mouse fibroblasts which expressed correspondingly mutated Omi/HtrA2 protein, an increased susceptibility of the cells to stress-induced apoptosis. The authors thereof did not recognize the possible significance of the Omi/HtrA2 protein or of the genetic modification described therein for Parkinson's disease. Moreover, the authors thereof investigated in further studies two families affected by Parkinson's disease for a corresponding mutation in the Omi/HtrA2 gene, but were unable to find one; cf. Jones et al., loc. cit., especially page 726, left-hand column, second paragraph.

The teaching perceived by the inventors therefore represents an abandonment of the opinion expressed to date in the art, not only not perceiving a connection of the Omi/HtrA2 gene with the pathological state of Parkinson's disease, but in fact denying it.

The inventors by contrast have been able to detect a genetic modification in the Omi/HtrA2 gene in 29 patients with Parkinson's disease, but not in more than 600 healthy controls. In parallel with this, the pathophysiological relevance of the detected genetic modifications have been perceived by the inventors by means of established toxicity tests.

The inventors were further able to show that the genetic modification results in damage to mitochondrial function.

Accordingly, detection of a nucleic acid molecule which encodes a genetically modified human Omi/HtrA2 protein in a human sample allows according to the invention a predisposition to or a pre-existing development of Parkinson's disease to be diagnosed. Moreover, since the significance of the Omi/HtrA2 gene for developing Parkinson's disease has been perceived for the first time, it is now possible to carry out targeted screening for further pathologically relevant genetic modifications in this gene.

A biological sample to be investigated according to the invention can be any biological material which contains representative nucleic acids and/or proteins of the corresponding human organism, for example a blood, tissue, saliva, hair or other sample.

Analysis of the biological sample in step (b) takes place by means of methods generally known in the art, for example by means of a mutation screening, in which, for example, PCR-based methods and heteroduplex analyses such as, for example, denaturing high-pressure liquid chromatography (dHPLC) or else hybridization techniques are used.

As the inventors have perceived, for successfully carrying out the method of the invention it is sufficient to investigate the biological sample for the presence of nucleic acid molecules which encode Omi/HtrA2 protein segments which have the genetic modification and preferably an appropriate amino acid residue present in position 141 or 399 in the complete protein. This is because, as the inventors have been able to find, a genetic modification precisely at this position in the Omi/HtrA2 complete protein is of crucial importance for the occurrence of Parkinson's disease.

It is preferred in the method according to the invention for the nucleic acid molecule to be detected to encode a human Omi/HtrA2 protein which has an amino acid exchange at amino acid position 141 or 399, further preferably has one through which an alanine molecule is exchanged for a serine molecule or a glycine molecule is exchanged for a serine molecule.

The analysis, which is preferred according to the invention, of the biological sample for the presence of such a nucleic acid molecule moreover has the advantage that a genetic modification or mutation which is extremely important and informative for correlation with Parkinson's disease is detected thereby. The inventors have surprisingly been able to show in this connection that 29 patients suffering from Parkinson's disease carry at least one of these said amino acid exchange mutations, whereas healthy controls showed no genetic modifications at all at position 399 or significantly more rarely at position 141. These detected genetic modifications are derived from nucleotide or base exchanges such as at positions 421 and 1195 of the open reading frame (ORF) of the human Omi/HtrA2 gene, wherein, compared with the wild type, a deoxyguanosine monophosphate (dGMP) or guanine (G) has been exchanged for deoxythymidine monophosphate (dTMP) or thymine (T) or a dGMP or G has been exchanged for a deoxyadenosine monophosphate (dAMP) or adenine (A).

Detection of such a nucleic acid molecule defined above by means of the method according to the invention therefore makes it possible for a predisposition to or a development of Parkinson's disease to be diagnosed reliably.

It is preferred in the method of the invention for the nucleic acid molecule to be detected to be one which binds under stringent conditions to the previously described nucleic acid molecule. In this connection, stringent conditions mean those conditions known to the skilled artisan under which only nucleic acid strands with perfect base pairing are formed and remain stable.

This measure has the advantage that, for example, the complementary non-coding strand of the human Omi/HtrA2 gene can also be used to diagnose Parkinson's disease, thus increasing the sensitivity of the method.

In this connection, it is further preferred in the method of the invention for the nucleic acid molecule to be detected to be one which under stringent conditions in turn binds to the nucleic acid molecule mentioned hereinbefore.

The advantage of this measure is that nucleic acid molecules which are derived from the ORF of the Omi/HtrA2 gene, such as, for example, mature or immature mRNA, and degradation products thereof, which still, however, harbor the characteristic genetic modification(s), and therefore bind to the nucleic acid molecule which is complementary to the nucleic acid molecule encoding the modified Omi/HtrA2 gene, are also used for the detection. This measure further increases the sensitivity of the method of the invention.

With this background, the present invention also relates to a nucleic acid molecule encoding a human Omi/HtrA2 protein which has, at amino acid position 141 and/or 399, a genetic modification compared with the wild type, preferably an amino acid exchange, further preferably one through which an alanine molecule is exchanged for a serine molecule at position 141 and/or a glycine molecule is exchanged for a serine molecule at position 399, and those nucleic acid molecules which code for corresponding segments thereof.

The present invention further relates to a nucleic acid molecule as explained, which binds under stringent conditions to the nucleic acid molecule described hereinbefore, and to a nucleic acid molecule likewise already explained, which binds under stringent conditions in turn to the latter.

On the basis of the relationship, perceived by the inventors for the first time, of a genetic modification in the Omi/HtrA2 gene, the present invention now enables to use the nucleic acid molecules described above for diagnosing Parkinson's disease and/or a predisposition thereto.

The present invention further relates to a host, preferably a transgenic non-human mammal, further preferably a transgenic mouse, a transgenic rat, a transgenic sheep, a transgenic goat or a transgenic cow, into which at least one nucleic acid molecule which encodes an Omi/HtrA2 protein which is genetically modified by comparison with the wild type, preferably one of the nucleic acid molecules described above, has been introduced.

The key role, described by various research groups, of the Omi/HtrA2 protein in caspase-independent apoptosis and in ubiquitin-dependent protein degradation, and the relationship, perceived for the first time by the inventors, between Omi/HtrA2 and Parkinson's disease are in favor of a key role of this gene and of the protein encoded thereby in the molecular pathogenesis of Parkinson's disease. Thus, neurodegenerative processes which usually do not comply with the classical criteria of caspase-mediated apoptotic or necrotic cell death are specifically also observed in Parkinson's disease. In addition, a disturbance of protein degradation which is shown by the formation of so-called Lewy bodies in the brain of affected patients is likewise described in Parkinson's disease. The mechanisms responsible for these phenomena have to date been unknown, despite intensive research. The finding of disease-causing mutations in the Omi/HtrA2 gene in Parkinson's disease by the inventors links together for the first time known pathogenic pathways of disturbed protein degradation to apoptotic processes. The inventors have further been able to detect in further experiments a ubiquitination of the Omi/HtrA2 protein and the latter in characteristic Lewy bodies in the brain of affected patients.

A model system with which the molecular pathology of Parkinson's disease can be replicated and analyzed more comprehensively than in the prior art is therefore provided by means of the transgenic mammal of the invention. Such a transgenic host also represents, of course, an excellent system for finding and testing substances effective for Parkinson's disease. The methodological steps necessary for producing a transgenic mammal such as, for example, an Omi/HtrA2 knock-out mouse, are described in detail in the art; cf. in this connection for example Thomas Rülicke (2001) Transgene, Transgenese, transgene Tiere: Methoden der nichthomologen DNA-Rekombination, Karger-Verlag.

The inventors have additionally perceived that diagnosis of Parkinson's disease is likewise possible after a modification of the novel method described above, namely if a (poly)peptide molecule which is derived from an Omi/HtrA2 protein which is genetically modified by comparison with the wild type is detected in the biological sample. The biological sample is preferably analyzed for the presence of a (poly)peptide molecule which is encoded by one of the nucleic acid molecules described above. The Omi/HtrA2 protein genetically modified as explained is the translation product of the correspondingly genetically modified nucleic acid molecule and therefore likewise provides direct information about a predisposition to or development of Parkinson's disease.

In a variant of the method of the invention, therefore, the biological sample is analyzed in step (b) for the presence of a (poly)peptide which is derived from an Omi/HtrA2 protein which is genetically modified by comparison with the wild type, preferably for the presence of a (poly)peptide which is encoded by one of the nucleic acid molecules described above.

This measure has the advantage that, if such a (poly)peptide is detected, an even firmer diagnosis of Parkinson's disease is possible, for example is possible even if the nucleic acid molecule encoding the peptide is undetectable or no longer detectable in the biological sample, e.g because of nuclease activities.

Detection of the (poly)peptide having the genetic modification, preferably at amino acid position 141 or 399, in the biological sample in this case takes place by protein purification and/or where appropriate sequencing methods known in the art. An overview of such methods is to be found for example in Lottspeich F. (editor) et al. (1998), “Bioanalytik”, Spektrum Akademischer Verlag.

Against this background, the present invention also relates to a (poly)peptide which is encoded by one of the nucleic acid molecules described above.

It will be appreciated that such a (poly)peptide also includes peptides which represent fragments, variants and isoforms of the genetically modified Omi/HtrA2 protein and which have the genetic modification explained above present at position 141 or 399 in the complete protein.

Such a (poly)peptide establishes the basis for developing pharmacologically active substances such as, for example, inhibitors or activators of the correspondingly genetically modified Omi/HtrA2 protein. The same applies to the nucleic acids encoding such a poly(peptide), by means of which for example pharmacologically interesting inhibiting RNAi(RNA interference) or siRNA(silencing RNA) molecules or other antisense molecules can be constructed. It is also possible by means of the coding nucleic acid molecules for example to produce the genetically modified Omi/HtrA2 protein in large quantities, to elucidate its three-dimensional structure and thereby to develop pharmacologically active substances by means of in silico screening.

It is preferred in the method of the invention described at the outset for the investigation in step (b) for the presence of the nucleic acid molecule of interest to take place by means of PCR technology.

This measure has the advantage that an extremely sensitive, established and substantially automatable method is used thereby, by means of which it is possible highly specifically to reach the genetically modified nucleic acid material which can then subsequently be detected by means of further standard methods such as electrophoresis/heteroduplex methods or direct sequencing.

The PCR primer preferably used is a nucleic acid molecule which comprises one of the sequences which is selected from the group consisting of: SEQ ID No. 1 to SEQ ID No. 17.

The inventors have surprisingly found that highly specific and selected amplification of the complete Omi/HtrA2 gene is possible by using the previously identified nucleic acid molecule comprising one of the listed nucleotide sequences, and therefore screening for genetic modifications is possible in the complete open reading frame. It has been possible to show that amplification of exon 1 of the human Omi/HtrA2 gene is possible with a PCR primer which has the nucleotide sequence SEQ ID No. 1 as forward primer, and with a PCR primer which has nucleotide sequences SEQ ID No. 2 as reverse primer. Correspondingly, it is possible with PCR primers with sequences SEQ ID No. 3 and SEQ ID No. 2 likewise to amplify exon 1, with sequences SEQ ID No. 4 and SEQ ID No. 5 to amplify exon 2, with sequences SEQ ID No. 6 and SEQ ID No. 7 to amplify exon 3, with sequences SEQ ID No. 8 and SEQ ID No.9 to amplify exon 4, with sequences SEQ ID No. 10 and SEQ ID No.11 to amplify exon 5, with sequences SEQ ID No. 12 and SEQ ID No. 13 to amplify exon 6, with sequences SEQ ID No. 14 and SEQ ID No. 15 to amplify exon 7, and with sequences SEQ ID No. 16 and SEQ ID No. 17 to amplify exon 8 of the Omi/HtrA2 gene.

It is then possible with the aid of the amplification products easily to detect a genetic modification which is present where appropriate using conventional mutation screening methods or hybridization methods.

Such an amplification is, of course, also possible with PCR primers which, besides one of the sequences SEQ ID No. 1 to SEQ ID No. 17, comprise 5′- and/or 3′-wards further nucleotides which hybridize where appropriate with the complementary strand, or which comprise minor sequence variations which, however, do not substantially alter the specificity of the primers. Nucleic acid molecules of this type are likewise encompassed by the use according to the invention as PCR primer pair.

Against this background, the present invention also relates to the corresponding use according to the invention of a nucleic acid molecule as PCR primer which binds under stringent conditions to a nucleic acid molecule which comprises one of the sequences which is selected from the group consisting of: SEQ ID No. 1 to SEQ ID No. 17.

A nucleic acid molecule which binds under stringent conditions to a nucleic acid molecule with one of the sequences SEQ ID No. 1 to SEQ ID No. 17 also makes possible the highly specific and selective amplification of a segment of the Omi/HtrA2 gene where appropriate genetically modified Omi/HtrA2 gene, although in this case hybridization takes place with the correspondingly complementary strand, but the amplicons are substantially identical.

On the basis of the unexpected properties and particular suitability of the novel nucleic acid molecules described above, the present invention also relates to the use thereof for amplifying the human Omi/HtrA2 gene.

It is preferred in the method of the invention explained above for the PCR amplicons to be analyzed by denaturing high pressure liquid chromatography (dHPLC) or other heteroduplex methods.

This method is one capable of selecting with high sensitivity sequence variants in PCR amplicons compared with the wild-type sequence. The detection is based on a heteroduplex which always forms after artificial denaturation and renaturation when the sequence variation in question is present on the second allele besides the wild-type Omi/HtrA2 allele. For this reason, to detect homozygous Omi/HtrA2 mutation the artificial denaturation by heating and slow cooling is preceded by additional admixture of “wild-type material”. In this case, during the preceding PCR, a heteroduplex is formed with higher reliability from a strand of the wild-type Omi/HtrA2 allele and a strand of the genetically modified Omi/HtrA2 allele. These hybrids show on a dHPLC column a retention behavior which is different from that of a homoduplex and can be detected with a probability of more than 95%.

The analysis time for a fragment is about four to five minutes, so that the dHPLC represents a very cost-effective and time-efficient screening method, for example preceding an additional sequencing of the amplicons. Information about this method is to be found for example in McCallum, C. M. et al., (2000), Targeted screening for induced mutations. Nature Biotechnology, 18, 455-457.

As alternative thereto, the genetic modification can be screened by the rapid direct sequencing method referred to as Pyrosequencing (concerning this, see www.pyrosequencing.com; the contents of the home page are incorporated in the present description by reference). It is also possible according to the invention to use the established restriction fragment length polymorphism analysis. Also suitable is single-strand conformation (SSCP) analysis, but this is less sensitive and more time-consuming in relation to the level of detection. It is also conceivable to carry out direct sequencing. This method is highly sensitive, but relatively costly and time-consuming.

It is preferred in the method of the invention for the analysis for the presence of the nucleic acid molecule in step (b) to take place by means of hybridization technology, preferably using as hybridization probe a nucleic acid molecule which has one of the sequences which is selected from the group consisting of SEQ ID No.18 to SEQ ID No. 21 shown in the appended sequence listing.

In a variant of the invention, the hybridization probe used is a nucleic acid molecule which binds under stringent conditions to a nucleic acid molecule identified above.

The inventors have been able in their studies to develop nucleic acid molecules through which, on use as hybridization probe, it is possible highly specifically to detect an exchange of deoxyguanosine monophosphate (dGMP) or guanine (G) for deoxythymidine phosphate (dTMP) or thymine (T) at position 421 of the open reading frame of Omi/HtrA2 gene, namely nucleic acid molecules which comprise the nucleotide sequence SEQ ID No. 18 or SEQ ID No. 19. This detection is, of course, also possible with complementary nucleic acid molecules which hybridize onto the corresponding antisense strand of the Omi/HtrA2 gene.

A nucleic acid molecule comprising the sequence SEQ ID No. 18 is moreover able to hybridize highly specifically onto the sense strand of exon 1 of the Omi/HtrA2 gene, whereas a nucleic acid molecule comprising a nucleotide sequence SEQ ID No. 19 can hybridize highly specifically onto the antisense strand of exon 1 of the Omi/HtrA2 gene.

As the inventors have been able to show further, a nucleic acid molecule which comprises the sequence SEQ ID No. 20 or SEQ ID No. 21 is suitable as hybridization probe with which an exchange of deoxyguanosine monophosphate (dGMP) or guanine (G) for deoxyadenosine monophosphate (dAMP) or adenine (A) at position 1195 of the open reading frame of the Omi/HtrA2 gene can be detected. The same applies to the correspondingly complementary nucleic acid molecules.

The nucleic acid molecule comprising the nucleotide sequence SEQ ID No. 20 binds highly specifically to the sense strand of exon 7, and the nucleic acid molecule having the nucleotide sequence SEQ ID No. 21 binds highly specifically to the antisense strand of exon 7 of the Omi/HtrA2 gene.

Because of the particular suitability, for example as PCR primer or hybridization probe, the present invention also relates to a nucleic acid molecule which comprises a nucleotide sequence which is selected from the group consisting of: SEQ ID No.1 to SEQ ID No. 21 according to the appended sequence listing, and a nucleic acid molecule which binds under stringent conditions to the above nucleic acid molecule.

The present invention further relates to a kit which comprises at least one of the nucleic acid molecules listed above.

Such a kit may, besides the nucleic acid molecules according to the invention, i.e. PCR primers or hybridization probes, comprise all the reagents, chemicals and buffer substances necessary for carrying out the method of the invention, plus a detailed description of the method of the invention or of another method which is to be carried out. This has the particular advantage that error-free work, especially by large laboratories with semi-skilled staff, is ensured thereby, i.e. manipulation errors for example on making up the buffers, in carrying out the method etc., are substantially avoided.

The present invention further relates to a method for finding substances which bind to human Omi/HtrA2 protein which is genetically modified by comparison with the wild type, which comprises the following steps (a) contacting a peptide which is derived from the genetically modified Omi/HtrA2 protein with a test substance under conditions which enable the test substance to bind to the peptide, and (b) establishing whether binding of the test substance to the peptide has taken place, where the genetic modification is an amino acid exchange at amino acid position 141 and/or at amino acid position 399 of the Omi/HtrA2 protein, by which an alanine molecule is exchanged for a serine molecule, or a glycine molecule is exchanged for a serine molecule.

The newly found correlation of Omi/HtrA2 protein which is genetically modified at amino acid position 141 or 399 with the clinical manifestation of Parkinson's disease makes the protein modified in this way a potential target for an aimed pharmacological intervention using selectively targeted substances. The novel method described above now makes it possible for the first time to find such substances which bind in a targeted manner to the modified Omi/HtrA2 protein and therefore have high therapeutic potential and substantially avoid side effects.

The inventors have perceived in this connection that provision of a peptide which is derived from the genetically modified Omi/HtrA2 protein but still has the genetically modified amino position 141 or 399 is sufficient for finding such substances. The method of the invention described above is additionally simplified thereby.

The substance to be tested may be in any conceivable chemical, biochemical or biological form, i.e. as a molecule such as a chemically defined compound, a peptide, protein, antibody, aptamer or as an ion or atom.

It is found by this novel method whether the test substance binds to the peptide, i.e. whether a state prevails in which the substance to be tested is located at least in the direct vicinity of the peptide, and therefore may possibly influence the activity of this peptide.

Step (b) takes place by means of molecular-biological and biochemical methods established in the art, for example affinity chromatographic or electrophoretic techniques.

Conditions enabling the test substance to bind to the peptide are sufficiently well known in the area of protein or enzyme biochemistry; these conditions are created for example by use of conventional physiological or biological buffer systems such as, for example, Tris, Hepes or PBS-based buffers, for example with the addition of various salts in suitable concentrations, and other conventional agents.

Because of the extremely significant pharmacological properties of such a substance found for a treatment of Parkinson's disease, the present invention also relates to a substance found by means of this method described above, and to a preferably pharmaceutical composition which comprises this substance and which comprises a pharmaceutically acceptable carrier and, where appropriate, further excipients. Suitable pharmaceutical carriers and excipients are known in the art; cf. Kibbe, A. H., (2000), “Handbook of Pharmaceutical Excipients”, 3rd edition, American Pharmaceutical Association and Pharmaceutical Press.

Further advantages and properties of the invention are evident from the following exemplary embodiments and the figures.

It will be appreciated that the features mentioned above and yet to be explained hereinafter can be used not only in the combinations indicated in each case but also in other combinations or alone without leaving the scope of the present invention.

The invention is now explained by means of exemplary embodiments and figures.

### BRIEF DESCRIPTION OF THE DRAWINGS

FIG. 1 shows the exon 1 mutation (G421T→A141S) found by dHPLC;

FIG. 2 shows the confirmation of the exon 1 mutation by direct sequencing;

FIG. 3 shows the confirmation of the exon 1 mutation by direct Pyrosequencing;

FIG. 4 shows the exon 7 mutation (G1195A→G399S) found by dHPLC;

FIG. 5 shows the confirmation of the exon 7 mutation by direct sequencing;

FIG. 6 shows the confirmation of the exon 7 mutation by RFLP analysis;

FIG. 7 shows the result of the cytotoxicity test using staurosporin,

FIG. 8 shows the result of the cytotoxicity test using MG132, and

FIG. 9 shows the result of a mitochondrion function test using staurosporin.

### DESCRIPTION OF PREFERRED EMBODIMENTS

### EXAMPLE 1

### Mutation Analysis of the Omi/HtrA2 Gene

Biological samples were taken from 411 or 514 patients suffering from Parkinson's disease and from more than 300 healthy controls, and the DNA was isolated therefrom by standard methods. The DNA was amplified by PCR. The PCR primers used for this are listed in Table I below.

TABLE I

| | |
|---|---|
| SEQ | SEQ |
| ID | ID |

| | | | | |
|---|---|---|---|---|
| Amplicon | Forward primer | No. | Reverse primer | No. |

| | | | | |
|---|---|---|---|---|
| Exon 1 | TGCCGCCTCTGAGTAGGG | 1 | CGGTCTACCCCCACCATT | 2 |
| Exon 1 | CCGGTTGTCTGTTGGGGT | 3 | CGGTCTACCCCCACCATT | 2 |
| | CA | | | |
| Exon 2 | TCTGTGCTTTCCCTCCAT | 4 | TCATCTGAAGATGCGAGC | 5 |
| | TT | | AA | |
| Exon 3 | GGTTGGAGCTGCTTATTT | 6 | TCCCCCATCATTGTCATT | 7 |
| | GC | | T | |
| Exon 4 | CCCAGACTTAGAATCCCC | 8 | GGGATTCTTGGAAGGAAG | 9 |
| | AGA | | GA | |
| Exon 5 | TAGGGAACTGGGGGCTGT | 10 | CCACATTAAAGGAACCCG | 11 |
| | AT | | TTT | |
| Exon 6 | GGCTCATTTGTCCCTCTG | 12 | CCCCCTCTGATTACACTG | 13 |
| | TC | | GT | |
| Exon 7 | GGGTTTGGCTAATAGGGT | 14 | CCATATCACACTGCAGCC | 15 |
| | GA | | TCT | |
| Exon 8 | TGTGTCCTTGAACTAGGC | 16 | GGAGCCTCATACTCTTGG | 17 |
| | TTTG | | TGA | |

A mutation screening was carried out with the amplicons. The mutation screening was carried using the dHPLC mutation detection system supplied by Transgenomic (WAVE). The dHPLC utilizes the difference in melting behavior of homoduplex and heteroduplex DNA. This entails double-stranded DNA being bound by means of TEAA (triethylammonium acetate) to an HPLC column and detached from the column by an increasing acetonitrile gradient. The DNA concentration of the acetonitrile buffer is detected by a laser after the column. The presence of heterozygous base exchanges on one strand with opposing bases in a double-stranded DNA strand leads, due to the instability at this site, to early detachment from the HPLC column, having the effect of shifting the detection peak on the WAVE system. The mutations can be detected through differences in the time of detachment of the DNA.

The employed DNA is in the form of PCR amplicon from patients or healthy controls, with each exon being amplified separately. In order to detect possible mutations of an exon as heteroduplex, the PCR product is denatured, and renatured again with slow cooling, before use in the WAVE apparatus. It is possible thereby for wild-type and mutated DNA strands to anneal and form heteroduplexes. Since homozygous mutations cannot be detected in this case, having regard to the relative rareness of Parkinson mutations the DNAs from two patients in each case were pooled together and then measured by dHPLC. Since conspicuous detection peaks are therefore always the result of two patients' samples, the respective patients' samples were measured again with reference DNAs (purchased from the “Centre d'études des polymorphismes humaines” (CEPH), Paris, France). The samples which were still conspicuous were directly sequenced in a Beckmann capillary sequencer.

The optimized dHPLC conditions for the developed PCR primer pairs are summarized in Table II below:

TABLE II

| PCR | Annealing | | Product | WAVE | |
|---|---|---|---|---|---|
| primer pair | temperature | MgCl₂ | length | temperature | WAVE gradient |
| (SEQ ID No.) | (° C.) | (mM) | (bp) | (° C.) | (% buffer B)* |
| 1 + 2 | 58 | 1 | 573 | 64.2 | 60-72 |
| 3 + 2 | 58 | 1 | 350 | 64.2 | 59-71 |
| 4 + 5 | 58 | 1 | 289 | 61.2 | 57-73 |
| | | | | 59.8 | 57-67 |
| 6 + 7 | 58 | 1 | 298 | 61.5 | 55-65 |
| | | | | 58.0 | 58-68 |
| 8 + 9 | 58 | 1 | 116 | 60.0 | 46-60 |
| 10 + 11 | 58 | 1 | 174 | 61.0 | 51-63 |
| 12 + 13 | 58 | 1 | 147 | 60.8 | 50-63 |
| | | | | 61.0 | 51-61 |
| 14 + 15 | 58 | 1 | 159 | 59.5 | 51-61 |
| | | | | | 50-66 |
| 16 + 17 | 58 | 1 | 251 | 60.5 | 56-66 |

*Buffer B = 0.1 M triethylammonium acetate (TEAA), 25% acetonitrile

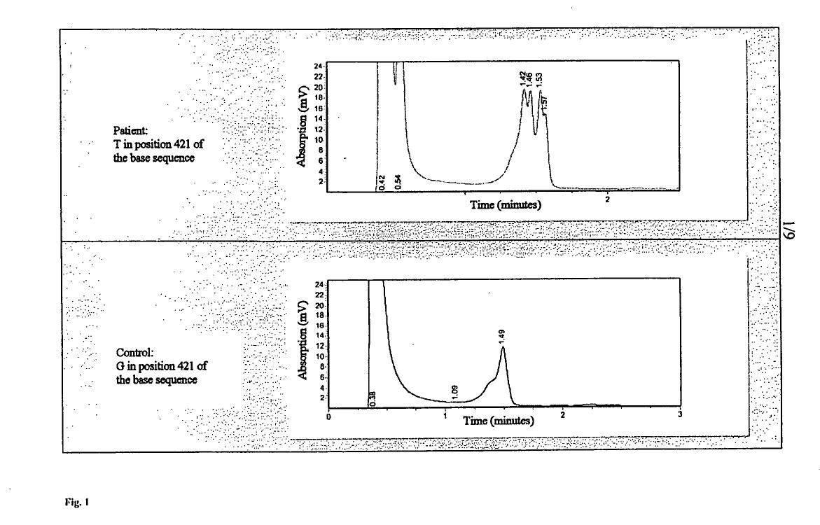

It was possible by this method to identify two mutations in the Omi/HtrA2 gene. The first mutation is a nucleotide exchange mutation in exon 1 of the gene, where a dGMP at position 421 of the open reading frame has been exchanged for a dTMP (cf. FIG. 1), leading at the protein level to an exchange of alanine for serine at position 141. This mutation was found in 25 of a total of 414 patients with Parkinson's disease, and exclusively in the heterozygous state.

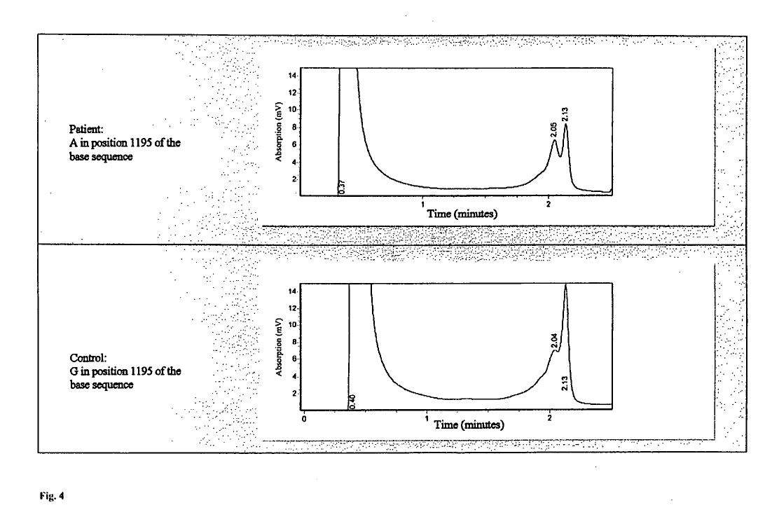

It was further possible to find a second mutation in the Omi/HtrA2 gene, where a dGMP at position 1195 of the open reading frame in exon 7 has been exchanged for a dAMP (cf. in this connection FIG. 4), resulting at the protein level in an exchange of glycine for serine at position 399. This mutation was found in 4 of a total of 514 patients with Parkinson's disease, and likewise exclusively in the heterozygous stage.

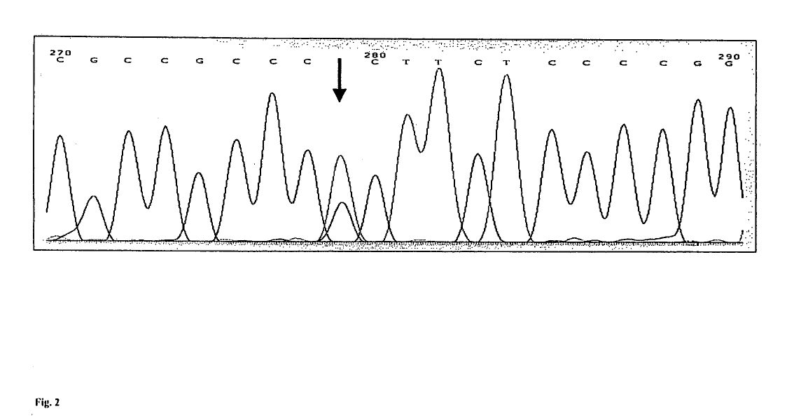

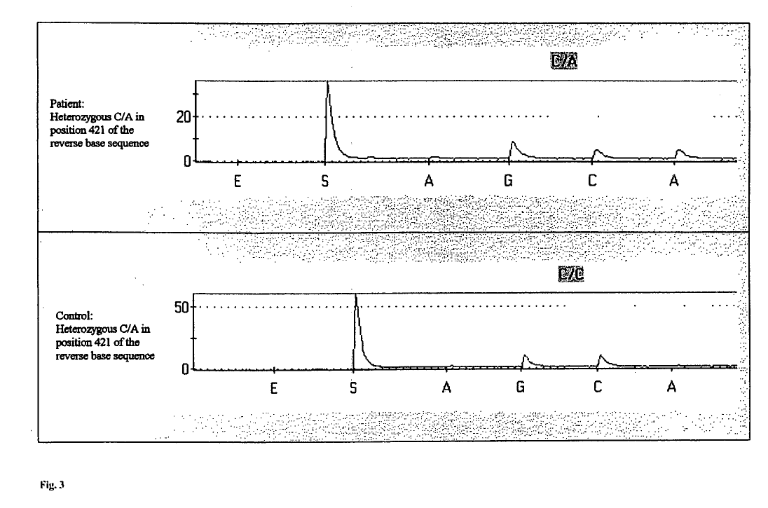

The presence of the mutation in exon 1 was verified by direct sequencing (cf. FIG. 2). It was further possible to confirm the presence of this mutation by Pyrosequencing (cf. FIG. 3); in this case, biotinylated variants of the PCR primers listed in Table I were employed.

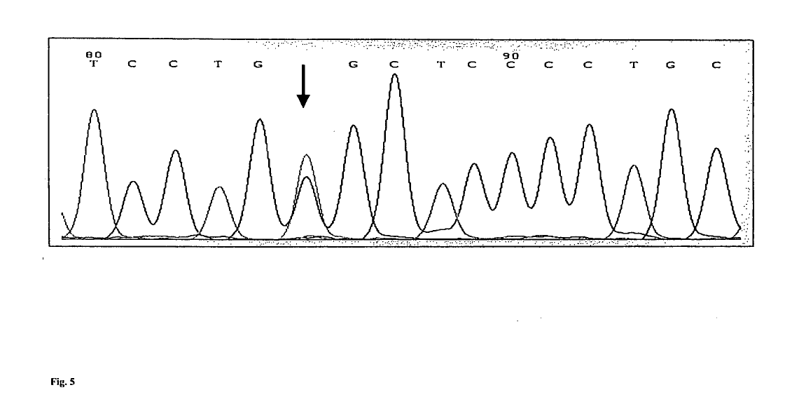

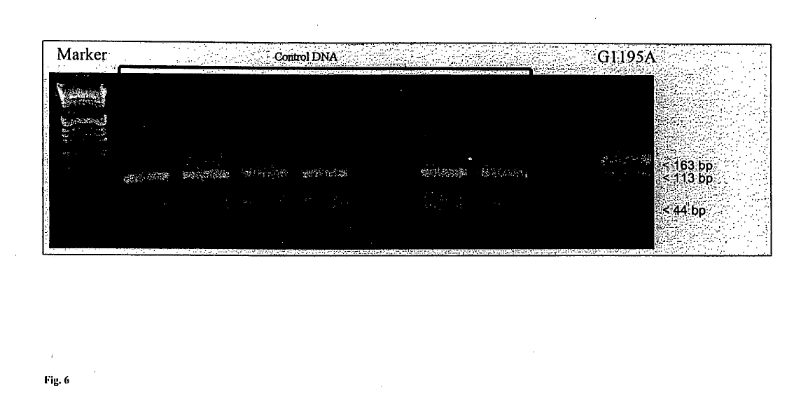

The nucleotide exchange mutation in exon 7 was likewise confirmed by direct sequencing (cf. FIG. 5) and also by restriction fragment length polymorphism(RFLP) analysis after MvaI digestion. With the wild-type sequence, restriction of the amplicon of exon 7 (163 base pairs) with MvaI results in three fragments: 113 bp, 44 bp and 6 bp. The G to A base exchange in position 1195 of the coding sequence leads to loss of a previously existing restriction cleavage site for the enzyme (cf. FIG. 6).

In order to determine the relevance of the two mutations found in relation to Parkinson's disease, chromosomes from healthy age-correlated controls were screened for the two mutations using the aforementioned methods. In this procedure, not one exon 7 mutation was detectable in 740 chromosomes from healthy controls. It was possible to find the exon 1 mutation in only 10 cases among 662 chromosomes of healthy controls (p=0.05).

The presence of one of the two found mutations accordingly correlates with a development of or predisposition to Parkinson's disease. Both mutations are therefore valuable diagnostic markers for Parkinson's disease.

### EXAMPLE 2

### Cytotoxicity Tests with Cells which Express Genetically Modified Omi/HtrA2 Protein

In order to analyze the pathogenetic relevance of the two mutations found in the Omi/HtrA2 gene, the cytotoxicity test was used to investigate whether cells which express the correspondingly genetically modified Omi/HtrA2 protein are more sensitive to cytotoxins, i.e. undergo apoptosis more frequently with corresponding exposure.

This test was carried out using the cytotoxicity detection kit (LDH) supplied by Roche. The measure of apoptosis used in this case is the lactate dehydrogenase (LDH) released from the cytosol of the apoptotic cells into the surrounding medium after contact with the toxins. This LDH in turn is determined via conversion of a tetrazolium salt into formazan, which can be measured through a change in color from yellow (tetrazolium salt) to red (formazan) using an ELISA reader at a wavelength of 490 nm. The absorption at 650 nm is measured as reference comparison.

In these investigations, 70 000 HEK293 cells (cells which stably express the wild-type Omi/HtrA2 protein or appropriately mutated Omi/HtrA2 protein; prepared by standard methods as described for example in Sambrook and Russell (2001), Molecular cloning—a laboratory manual, Cold Spring Harbor Laboratory Press, New York, the contents of which are included in the description by reference) in each kit were seeded in a 24-well plate. The cells were incubated under normal culturing conditions in DMEM (Invitrogen), 10% inactivated fetal calf serum (Invitrogen), 1% penicillin/streptomycin (Invitrogen) for 24 hours. The culture medium was then removed and replaced by LDH assay medium (DMEM), 1% inactivated fetal calf serum, penicillin/streptomycin, which contained as cytotoxin either 0.5 μM staurosporin or 3.0 μM MG132 or solvent as negative control. The test was carried out in this case as triplicate test (in each case for toxin-exposed cell lines and controls). The appropriate medium was transferred into an empty well and incubated with the cells as background value for the ELISA reader.

Incubation on use of MG132 was for 24 hours, and incubation on use of staurosporin was for 6 hours. After these incubation times, 150 μl were removed from each well and centrifuged. The centrifugation step served to sediment detached cells which might have altered the real LDH concentration through a later apoptosis. After the centrifugation, 100 μl of the supernatant were transferred into a 96-well plate. The remaining 50 μl were resuspended and returned to the corresponding wells of the 24-well plate.

In order to detect the total amount of LDH, which is related to the total number of cells per well, then 50 μl of 10% Triton X-100 solution were pipetted into each well in order to lyse the cells which were still alive. After an incubation time of 20 minutes, 100 μl of the centrifuged supernatant were then in turn pipetted into the 96-well plate. 100 μl of LDH assay medium were then put in each well to be measured of the 96-well plate and incubated at room temperature for 20 minutes.

After this incubation time had elapsed, the reaction was stopped by adding 50 μl of 1 N HCl (Sigma) and measured using the ELISA reader. The LDH value then results from the ratio of the absorption of the first and second supernatant of a well and can thus be used as value for the proportion of cells which have died. The average of three wells treated in the same way yields the proportion of dead cells for a measurement with standard deviation.

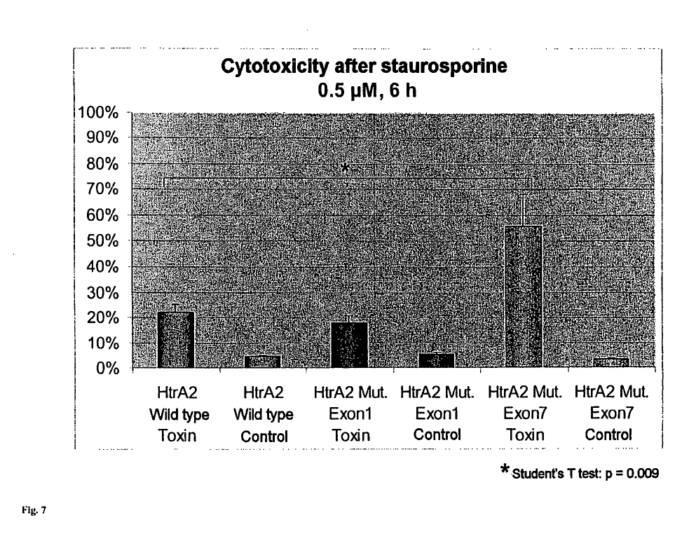

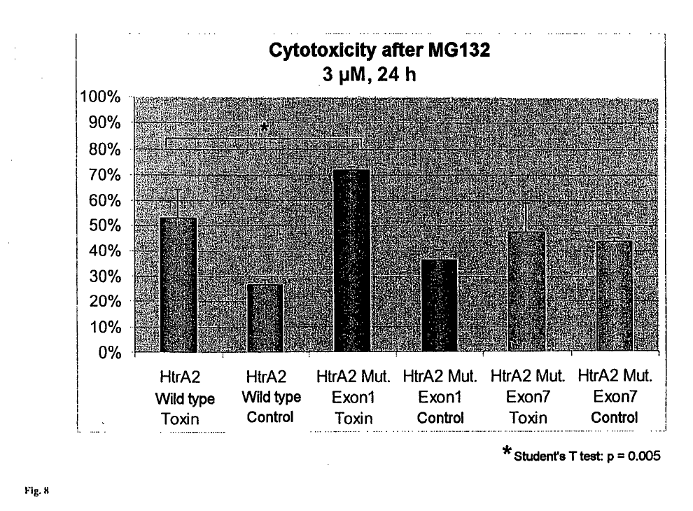

The result of such an experiment is depicted in FIGS. 7 and 8. These show that the HEK293 cells which expressed the exon 7, i.e. the G1195A, mutation (G399S) were more than five times more sensitive to staurosporin than the wild-type HEK293 cells; FIG. 7, cf. first bar with fifth bar from the left.

It further emerges that the HEK293 cells which expressed the exon 1, i.e. G421T, mutation (A141S) were approximately 1.4 times more sensitive to MG132 than wild-type HEK293 cells; FIG. 8, cf. first bar with third bar from left.

### EXAMPLE 3

### Function Test on Mitochondria from Cells which Express Genetically Modified Omi/HtrA2 Protein

Because of the observed morphological changes in the mitochondria in cells with overexpression of mutated Omi/HtrA2, the mitochondrial function in these cells was investigated.

The mitochondrial membrane potential was measured by FACS analysis (flow cytometer) using JC-1, as marker of mitochondrial homeostasis in SH-SY5Y cells which stably express the wild-type or mutated (S141 or S399) Omi/HtrA2.

JC-1 is a green fluorescent dye which can be employed to measure the mitochondrial membrane potential. It has the property of diffusing through cellular membranes and is moreover in the form of a monomer which fluoresces green on excitation with a 488 nm laser. In intact mitochondria there is accumulation of the dye owing to the mitochondrial membrane potential, and thus JC-1 aggregates form. The aggregate now re-emits in a red fluorescent wavelength the excitation light and can thus be clearly distinguished both under a microscope and in a flow cytometer from the green fluorescent monomers. It is thus possible with JC-1 to measure the mitochondrial membrane potential of mitochondria very well and also make statements about the early onset of apoptosis in cells, because the membrane potential collapses at a very early time during apoptosis.

Staurosporin was used as model of cellular stress in order to provoke loss of the mitochondrial membrane potential.

The JC-1 assay was adapted according to the description of the dye by Molecular Probes. For this purpose, cells were seeded in 6-well plates 24 hours before toxin exposure (HEK293: 700 000 cells/well, SH-SY 5H: 1 000 000 cells/well) and allowed to grow adherently for 24 hours. After this time, the cells were cultivated further with the appropriate toxins in their normal cell culture medium and, after various incubation times, used further for the analysis.

The cells were then detached from the cell culture dish with trypsin, centrifuged (1200 rpm, 4 minutes) and washed once in PBS. After washing, recentrifuged and then resuspended in 500 μl of a 5 μg/ml concentrated JC-1/PBS solution. The cells were then incubated at 37° C. for 30 minutes. The incubation with JC-1 was followed by three washing steps with PBS, immediately followed by analysis in the flow cytometer.

In order to differentiate the cell population between green and red fluorescence and to make the adjustment in the flow cytometer, cells without JC-1, with JC-1 and cells with JC-1 treated with CCCP were used. CCCP is a protonophore which reversibly abolishes the mitochondrial membrane potential. It was thus possible after the staining readily to excite only the green fluorescence of the JC-1 monomers, owing to treatment with CCCP. After adjustment of the assay in the flow cytometer, the samples were then measured and evaluated.

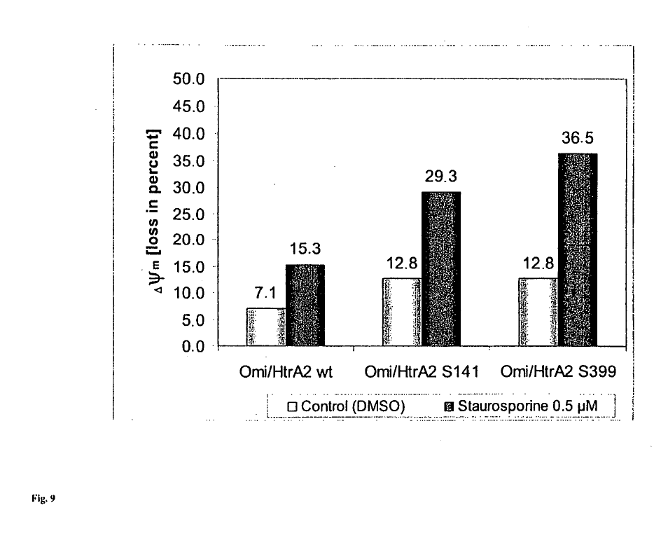

The result of this experiment is depicted in FIG. 9. In this case, the various cells were treated with 0.5 μm staurosporin for 4 hours.

The y axis indicates the decline in the membrane potential in percent (ΔΨm [loss in percent]). The x axis depicts on the left the result of the measurements on wild-type cells (Omi/HtrA2 wt), in the middle on cells with the exon 1 mutation (Omi/HtrA2 S141), and on the right on cells with the exon 7 mutation (Omi/HtrA2 S399).

The pale bar on the left in each case shows the result of the measurements on untreated or dimethyl sulfoxide-treated cells (control (DMSO)), and the dark bar on the right in each case shows the result of the measurements on cells treated with 0.5 μM staurosporin (staurosporin 0.5 μM).

Analysis of the JC-1 fluorescence in this case showed that there is a distinct decline in the mitochondrial membrane potential after treatment with staurosporin in cells which have the S141 and S399 mutations in the Omi/HtrA2 protein compared with wild-type cells.

The mutations found in the Omi/HtrA2 protein therefore lead to impairment of mitochondrial functional.

The two newly found mutations in the human Omi/HtrA2 gene accordingly are genetic modifications which have considerable effects on the integrity of the cells. Incubation of such genetically modified cells with cytotoxin leads to an increased onset of apoptosis thereof. The inventors have thus found for the first time genetic modifications associated with Parkinson's disease which are involved both in the disturbed protein degradation—the Omi/HtrA2 protein is a constituent of pathognomonic protein aggregates, called Lewy bodies, as the inventors were able to confirm by their own experiments—and in the regulation of apoptosis, as shown by the data discussed above.

The provision of the teaching of the invention therefore not only provides a diagnostic tool for discovering the development of or predisposition to Parkinson's disease, but also creates the basis for the development of a model system by means of which for example novel therapeutically effective antiparkinson substances can be discovered, or the molecular-pathological bases of the development of Parkinson's disease can be understood better.

Therefore, what is claimed is:

1. A method for diagnosing Parkinson's disease in a human being, comprising the following steps:
(a) providing a biological sample of the human being;
(b) analysing the biological sample for the presence of a nucleic acid molecule and/or of a (poly)peptide, and
(c) correlating a positive finding with the development of Parkinson's disease and/or with a predisposition to the development of Parkinson's disease,
wherein in step (b) the nucleic acid molecule encodes an Omi/HtrA2 protein which is genetically modified compared with the wild type, or segments thereof, or in that the (poly)peptide is derived from an Omi/HtrA2 protein which is genetically modified compared with the wild type.

2. The method of claim 1, wherein through said genetic modification at amino acid position 141 and/or at amino acid position 399 of the Omi/HtrA2 protein an amino acid exchange has taken place.

3. The method of claim 2, wherein through said genetic modification at amino acid position 141 of the Omi/HtrA2 protein an alanine molecule is exchanged for a serine molecule, and/or at amino acid position 399 of the Omi/HtrA2 protein a glycine molecule is exchanged for a serine molecule.

4. The method of claim 1, wherein the analysis for the presence of the nucleic acid molecule in step (b) is performed by means of PCR technology.

5. The method of claim 4, wherein a nucleic acid molecule used as PCR primer comprises one of the sequences which is selected from the group consisting of: SEQ ID No. 1 to SEQ ID No. 17 according to the appended sequence listing.

6. The method of claim 4, wherein a nucleic acid molecule used as PCR primer binds under stringent conditions to a nucleic acid molecule which comprises one of the sequences which is selected from the group consisting of: SEQ ID No. 1 to SEQ ID No. 17 according to the appended sequence listing.

7. The method of claim 4, wherein the PCR amplicons are analyzed by denaturing high pressure liquid chromatography (dHPLC), heteroduplex methods or direct sequencing.

8. A nucleic acid molecule which comprises one of the sequences which is selected from the group consisting of: SEQ ID No. 1 to SEQ ID No. 17 according to the appended sequence listing.

9. A nucleic acid molecule which binds under stringent conditions to the nucleic acid molecule of claim 8.

10. The method of claim 1, wherein the analysis for the presence of the nucleic acid molecule in step (b) is performed by means of hybridization technology.

11. The method of claim 10, wherein a nucleic acid molecule used as hybridization probe comprises one of the sequences which is selected from the group consisting of: SEQ ID No. 18 to SEQ ID No. 21 according to the appended sequence listing.

12. The method of claim 10, wherein a nucleic acid molecule used as hybridization probe binds under stringent conditions to a nucleic acid molecule which comprises one of the sequences which is selected from the group consisting of: SEQ ID No. 18 to SEQ ID No. 21 according to the appended sequence listing.

13. A nucleic acid molecule which comprises one of the sequences which is selected from the group consisting of: SEQ ID No. 18 to SEQ ID No. 21 according to the appended sequence listing.

14. A nucleic acid molecule which binds under stringent conditions to the nucleic acid molecule of claim 13.

15. A kit which comprises at least one nucleic acid molecule comprising one of the sequences which is selected from the group consisting of: SEQ ID No. 1 to SEQ ID No. 21 according to the appended sequence listing.

16. A method for finding substances which bind to human Omi/HtrA2 protein which is genetically modified by comparison with the wild type, which comprises the following steps:
(a) contacting a peptide which is derived from the genetically modified Omi/HtrA2 protein with a test substance under conditions which enable the test substance to bind to the peptide, and
(b) establishing whether binding of the test substance to the peptide has taken place,
wherein the genetic modification is an amino acid exchange at amino acid position 141 and/or at amino acid position 399 of the Omi/HtrA2 protein, by which an alanine molecule is exchanged for a serine molecule, or a glycine molecule is exchanged for a serine molecule.

17. A substance found by the method of claim 16.

18. A composition which comprises the substance of claim 17.

19. The composition as claimed in claim 18, which is a pharmaceutical composition and comprises a pharmaceutically acceptable carrier and, where appropriate, further excipients.

20. A nucleic acid molecule encoding a human Omi/HtrA2 protein which has a genetic modification at amino acid position 141 and/or 399 compared with the wild type, and corresponding segments thereof.

21. The nucleic acid molecule of claim 20, wherein the genetic modification is an amino acid exchange.

22. The nucleic acid molecule of claim 21, wherein through the amino acid exchange an alanine molecule is exchanged for a serine molecule at position 141 and/or through the amino acid exchange a glycine molecule is exchanged for a serine molecule at position 399.

23. A nucleic acid molecule which binds under stringent conditions to the nucleic acid molecule of claim 20.

24. A nucleic acid molecule which binds under stringent conditions to the nucleic acid molecule of claim 23.

25. A host into which at least one nucleic acid molecule which encodes an Omi/HtrA2 protein which is genetically modified by comparison with the wild type has been introduced, wherein said host is selected from the group consisting of: a transgenic non-human mammal, a transgenic mouse, a transgenic rat, a transgenic sheep, a transgenic goat and a transgenic cow.

26. A (poly)peptide encoded by the nucleic acid molecule of claim 20.

27. A (poly)peptide encoded by the nucleic acid molecule of claim 22.


SEQUENCE LISTING (23 sequences)

SEQ ID NO: 1 - DNA, 18 bp, Artificial Sequence
  PCR primer
tgccgcctctgagtaggg

SEQ ID NO: 2 - DNA, 18 bp, Artificial Sequence
  PCR primer
cggtctacccccaccatt

SEQ ID NO: 3 - DNA, 20 bp, Artificial Sequence
  PCR primer
ccggttgtctgttggggtca

SEQ ID NO: 4 - DNA, 20 bp, Artificial Sequence
  PCR primer
tctgtgctttccctccattt

SEQ ID NO: 5 - DNA, 20 bp, Artificial Sequence
  PCR primer
tcatctgaagatgcgagcaa

SEQ ID NO: 6 - DNA, 20 bp, Artificial Sequence
  PCR primer
ggttggagctgcttatttgc

SEQ ID NO: 7 - DNA, 19 bp, Artificial Sequence
  PCR primer
tcccccatcattgtcattt

SEQ ID NO: 8 - DNA, 21 bp, Artificial Sequence
  PCR primer
cccagacttagaatccccaga

SEQ ID NO: 9 - DNA, 20 bp, Artificial Sequence
  PCR primer
gggattcttggaaggaagga

SEQ ID NO: 10 - DNA, 20 bp, Artificial Sequence
  PCR primer
tagggaactgggggctgtat

SEQ ID NO: 11 - DNA, 21 bp, Artificial Sequence
  PCR primer
ccacattaaaggaacccgttt

SEQ ID NO: 12 - DNA, 20 bp, Artificial Sequence
  PCR primer
ggctcatttgtccctctgtc

SEQ ID NO: 13 - DNA, 20 bp, Artificial Sequence
  PCR primer
ccccctctgattacactggt

SEQ ID NO: 14 - DNA, 20 bp, Artificial Sequence
  PCR primer
gggtttggctaatagggtga

SEQ ID NO: 15 - DNA, 21 bp, Artificial Sequence
  PCR primer
ccatatcacactgcagcctct

SEQ ID NO: 16 - DNA, 22 bp, Artificial Sequence
  PCR primer
tgtgtccttgaactaggctttg

SEQ ID NO: 17 - DNA, 21 bp, Artificial Sequence
  PCR primer
ggagcctcatactcttggtga

SEQ ID NO: 18 - DNA, 28 bp, Artificial Sequence
  hybridization probe
ctagcccgccgccctcttctccccggag

SEQ ID NO: 19 - DNA, 28 bp, Artificial Sequence
  hybridization probe
ctccggggagaagagggcggcgggctag

SEQ ID NO: 20 - DNA, 31 bp, Artificial Sequence
  hybridization probe
ccataaagtcatcctgtgctcccctgcacac

SEQ ID NO: 21 - DNA, 30 bp, Artificial Sequence
  hybridization probe
ccggtgtgcaggggagcacaggatgacttt

SEQ ID NO: 22 - DNA, 2187 bp, Homo sapiens
cgtggatcccgagaaagaggcgcaggacgaggaggcagaacccgactggcgcgtagagcagcagcacgagcagtaggaagcagtcacccggaagcctgggggcgagaggcgaagtggtcaggcgccgaaggccgagagcacgcggggatcggtctcttcccgccgggtctcttaccggtgcgagtcaaagagccgctccggccccggccctgagggaagctccataactgctgcttcaggagcgcccggccgtcgccgccgccgccattttcgcgcccggccgcaggggctcttgggaaggcggagtctttgggcatccgcccggggtgaggggacccgaagtcctgaggcgcgccggaagggctagcggtcccagcataccccgcggccccttgggccgtctcacaactcgcgtccggcggagaccacaattcccggcattcgtggggcagggaggagtcggcctcccggaatcctggtcccggcgtgcacttctgaaggacttcaggtaccggcgtgccccgcgtcctactgtccgcctgctcgcgtcctgggtgccgcctctgagtagggcgggcgaggaggcagccaaggcggagctgatggctgcgccgagggcggggcggggtgcaggctggagccttcgggcatggcgggctttggggggcattcgctgggggaggagaccccgtttgacccctgacctccgggccctgctgacgtcaggaacttctgacccccgggcccgagtgacttatgggacccccagtctctgggcccggttgtctgttggggtcactgaaccccgagcatgcctgacgtctgggaccccgggtccccgggcacaactgactgcggtgaccccagataccaggacccgggaggcctcagagaactctggaacccgttcgcgcgcgtggctggcggtggcgctgggcgctgggggggcagtgctgttgttgttgtggggcgggggtcggggtcctccggccgtcctcgccgccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaatgaatagatcaccaagagtatgaggctcctgctctgatttcctccttgcctttctggctgaggttctgagggcaccgagacagagggttaaatgaaccagtgggggcaggtccctccaaccaccagcactgactcctgggctctgaagaatcacagaaacactttttatataaaataaaattatacctagcaacataaaaaaaaaaaaaaa

SEQ ID NO: 23 - protein, 458 aa, Homo sapiens
MAAPRAGRGAGWSLRAWRALGGIRWGRRPRLTPDLRALLTSGTSDPRARVTYGTPSLWARLSVGVTEPRACLTSGTPGPRAQLTAVTPDTRTREASENSGTRSRAWLAVALGAGGAVLLLLWGGGRGPPAVLAAVPSPPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLHRGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYVTPEVTE